(19) United States
(12) Patent Application Publication
(10) Pub. No.: US 2014/0039524 A1
(43) Pub. Date: Feb. 6, 2014

(54) SURGICAL CLIP APPLICATOR

(71) Applicant: Aesculap AG, Tuttlingen (DE)

(72) Inventors: Peter Schulz, Loeffingen (DE); Pedro Morales, Tuttlingen (DE); Dieter Weisshaupt, Immendingen (DE)

(73) Assignee: Aesculap AG, Tuttlingen (DE)

(21) Appl. No.: 13/960,892

(22) Filed: Aug. 7, 2013

Related U.S. Application Data
(63) Continuation of application No. PCT/EP2012/054107, filed on Mar. 9, 2012.

(30) Foreign Application Priority Data
Mar. 31, 2011 (DE) 10 2011 001 705

(51) Int. Cl. A61B 17/128

(52) U.S. Cl. CPC A61B 17/1285
USPC 606/142

(57) ABSTRACT

A surgical clip applicator for U-shaped or V-shaped clips having two legs connected to each other by a connecting region is provided. In order to enable application and secure closure of the clips with a reduced expenditure of force, the applicator, which has a handle, an adjoining shaft and at the free end of the shaft an applying tool, which is actuatable with the handle and has two tool jaws which are transferable from an open, idle position to a closed position when actuated, is configured with receiving areas for the clip to be applied. These receiving areas each have an abutment surface for one of the legs, which extends at least over part of the length of the legs, and a recess in which the connecting region of the legs of the clip is arranged when the tool jaws are in the closed position.

This application is a continuation of international application number PCT/EP2012/054107 filed on Mar. 9, 2012 and claims the benefit of German application number 10 2011 001 705.4 filed on Mar. 31, 2011, which are incorporated herein by reference in their entirety and for all purposes.

### BACKGROUND OF THE INVENTION

The invention relates to a surgical clip applicator for generally U-shaped or V-shaped clips comprising two legs connected to each other by a connecting region. The surgical clip applicator comprises a handle, a shaft adjoining the handle, and an applying tool for the clips at the free end of the shaft, which is actuatable with the handle and comprises two tool jaws transferable from an open, idle position to a closed position when actuated. The tool jaws each comprise a receiving area for the clip to be applied, and the clips are often kept ready in a clip magazine which is held, for example, on the shaft of the clip applicator.

Such a surgical clip applicator is known, for example, from DE 19603889 A1.

In addition to simple U-shaped or V-shaped clips, so-called double-shank clips or multiple clips are also known in which two or three clips in parallel alongside one another are brought in one work step into contact with a tissue structure, for example, with a blood vessel.

With the known clip applicators there is the problem, particularly when double- shank clips or multiple clips are used, that the force for closing the clips, which merely on account of the clip material has to be exerted in order to securely close the clips or securely bring the clips into contact with the tissue structure, is considerable and requires a corresponding expenditure of force on the part of the surgeon performing the operation.

The object of the present invention is to propose a surgical clip applicator of the kind mentioned at the outset, which enables application and secure closure of the clips with a reduced expenditure of force.

### SUMMARY OF THE INVENTION

This object is accomplished by a surgical clip applicator having the features of claim 1.

Owing to the recess provided in the abutment surfaces of the tool jaws for receiving the connecting region of the clip, the clip or its legs can be securely brought into contact with the tissue structure, for example, the blood vessel, without the connecting region also having to be completely compressed. With the clip applied with the surgical clip applicator in accordance with the invention, there remains in the connecting region a small open area which is typically filled either by tissue structure, for example, the wall of a blood vessel, or may remain empty and does not impede safe closure of a blood vessel.

The clip, therefore, undergoes deformation to a lesser extent in the connecting region, with the result that the expenditure of force for closing the clip is reduced, while the legs are closed to such an extent that the required dimension of the gap, for example, approximately 0.25 mm or less, in particular, approximately 0.07 to approximately 0.15 mm, is maintained.

This saving of force with deformation of the clip in order to close it is twice as great or several times greater when double-shank clips or multiple clips are used.

As a result, working with the surgical clip applicator in accordance with the invention is considerably easier, so that more extensive work is also considerably less tiring, while the required dimension of the gap of the closed clips can be safely achieved.

The abutment surfaces of the surgical clip applicators in accordance with the invention may differ in configuration. In one embodiment, the abutment surfaces extend in a uniformly planar manner from the free ends of the legs to the recess which receives the connecting region of the legs of the clip. Here an abutment surface is created, which brings the legs of the clips from their free end to the connecting region uniformly into contact with the tissue structure and thus ensures as planar an alignment of the two legs in the applied state as possible.

In an alternative embodiment, it may be provided that in the clip applicator in accordance with the invention, the abutment surface comprises a setback in the region of the free ends of the legs, so that the legs of the clip are safely gripped in the middle area, i.e., in the area between the free ends and the connecting region and bought into contact with the tissue structure to which the clip is to be applied.

This is possible because on account of the V-shaped or U-shaped clip structure, a pivoting of the legs of the clip occurs about an axis defined by the connecting region via the abutment surfaces, with the clip material undergoing plastic deformation in the connecting region. Owing to this deformation, application of force in the middle area of the legs is sufficient to ensure secure contact of the entire legs with the tissue structure, in particular, with a blood vessel.

The abutment surfaces of a clip applicator in accordance with the invention are preferably of such dimensions that the legs abut against the tool jaws over approximately a third of their length or more, in particular, half of the length or more, when the tool jaws are in the closed position.

In a further preferred embodiment, the abutment surfaces are of such dimensions that the legs abut against the abutment surfaces over approximately two thirds of their length or more when the tool jaws are in the closed position.

The recesses set back from the receiving areas of the tool jaws may differ in configuration. The cross section, seen perpendicularly to the longitudinal direction of the legs of the clips, may be semicircular or polygonal, with the resulting setback in relation to the abutment surfaces preferably being such, in each case, that no force is exerted on the connecting region of the clip when the tool jaws are in the closed position.

Here the connecting region is preferably defined as a distance from the apex of the clip in the direction towards the free end of a leg of the clip material, which is approximately four times or less, in particular, approximately three times the diameter or thickness of a clip leg.

In this context, thickness of a clip leg is understood as its extent in the closing direction of the clip.

The depth of the recess is preferably approximately half of the diameter or thickness of a clip leg or more.

In particular, the depth of the recess is approximately equal to the thickness or the diameter of a clip leg.

In a preferred clip applicator in accordance with the invention, the recesses extend in the direction of the longitudinal direction of the legs of the clips over a length which corresponds approximately to twice the diameter or thickness of a clip leg or more.

Further preferred, the length of the recess is approximately four times the diameter or thickness of the clip leg or less. This ensures that also under load the clip remains in its intended receiving position in the applying tool and is unable to inadmissibly move away in the proximal direction.

The total length of the receiving area for the clip in the tool jaws preferably corresponds approximately to the length of the clip legs from the apex of the V-shaped or U-shaped clip to the respective free end measured along the course thereof. This length is greater than the longitudinal extent of the clip and is not to be confused with it.

While the receiving area preferably forms a stop for the connecting region (apex) of the clip at the proximal end, a stop for the free ends of the clip legs is not provided in all cases at the distal end of the receiving area.

Furthermore, at least in the area of the recesses receiving the connecting regions, the receiving areas are preferably widened or open at the sides, i.e., in the transverse direction in relation to the clip plane, so that the bulge of material which is created in the connecting regions when the clip is closed can form there without restraints.

Further preferred, the abutment surfaces of the tool jaws are provided with a guide element.

In the case of single clips, the guide elements may be formed as grooves, the depth of which is somewhat less than the thickness or diameter of the clip legs.

Alternatively or additionally, the guide element may have an elongate projection, in particular, when double-shank or multiple clips are used. These elongate projections are then arranged parallel to the longitudinal direction of the abutment surfaces for the clip legs, so that the double-shank and multiple clips remain oriented and guided in parallel with their plurality of legs when the tool jaws are transferred from their open, idle position to the closed position.

The applying tool may be of forceps-type configuration with tool jaws mounted for pivotal movement in relation to each other. Alternatively, an applying tool of fork-shaped configuration is possible, in which the tool jaws are arranged at free ends of elastically deformable fork prongs as shown, for example, in DE 2845213.

These and further advantages of the invention will be explained in greater detail hereinbelow with reference to the drawings.

### BRIEF DESCRIPTION OF THE DRAWINGS

FIG. 1 shows a clip applicator in accordance with the invention in sectional representation;

FIG. 2 shows a detail of the clip applicator from FIG. 1;

FIGS. 3A-3D show a schematic representation of the operation of closing a clip using a clip applicator in accordance with the invention;

FIGS. 4A-4C show a detail of a surgical clip applicator in accordance with the invention during the operation of applying a clip;

FIG. 4D shows a clip closed with a clip applicator in accordance with the invention; and

FIGS. 5A and 5B show alternative embodiments of a surgical clip applicator in detail.

### DETAILED DESCRIPTION OF THE INVENTION

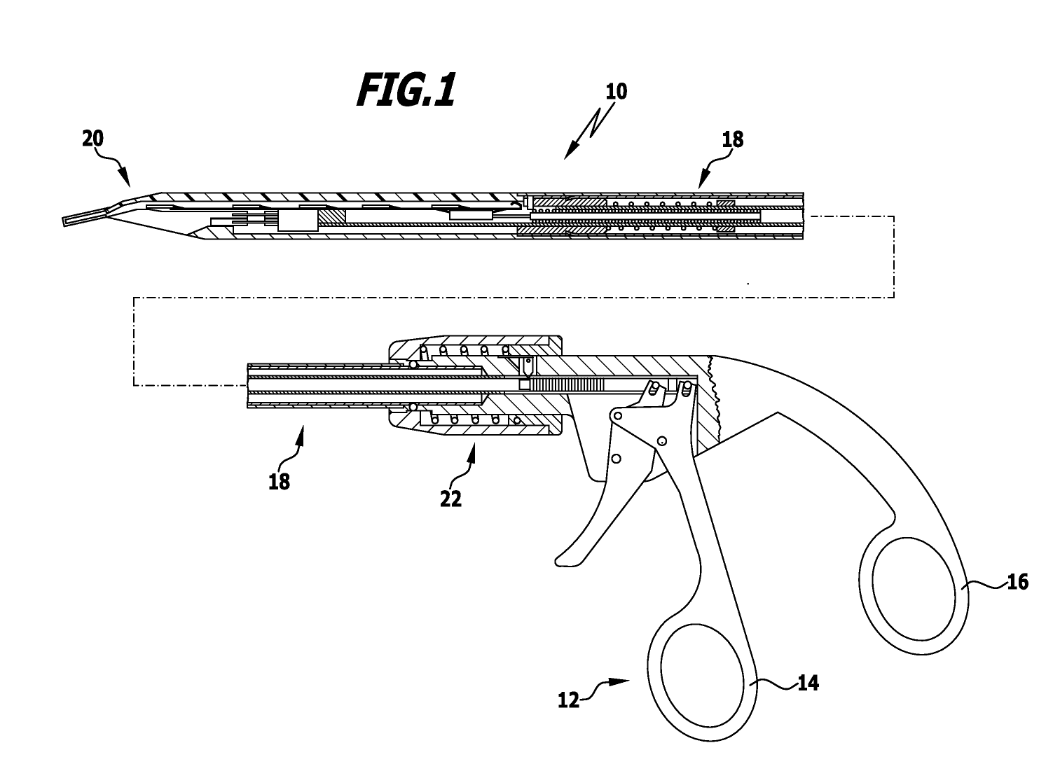

FIG. 1 shows a surgical clip applicator in accordance with the invention, which is based in its fundamental structure on the clip applicator described in DE 19603889 A1 in conjunction with the FIG. 1 shown therein.

The clip applicator 10 comprises a handle 12 with two handle parts 14, 16, an adjoining shaft 18 and an applying tool 20 arranged at the free end of the shaft 18. The handle 12 is preferably held rotatably and releasably on the shaft 18 by means of a coupling piece 22.

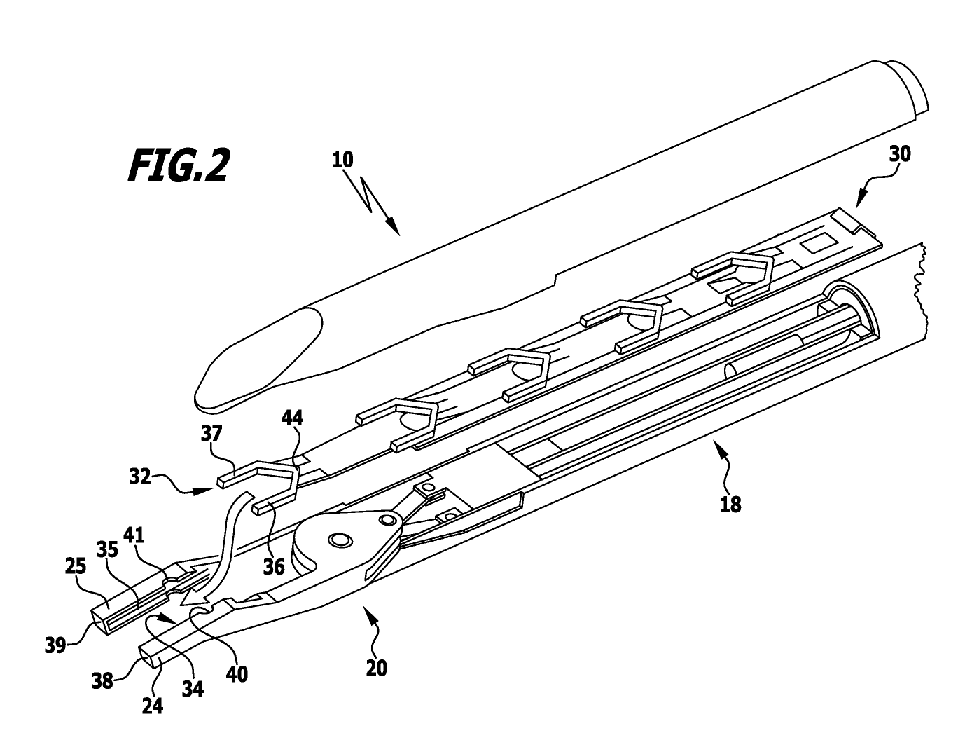

In the embodiment of the surgical clip applicator 10 in accordance with the invention shown in FIGS. 1 and 2, the applying tool 20 arranged at the free end of the shaft 18 is of forceps-type configuration and comprises two tool jaws 24, 25 mounted for pivotal movement in relation to each other. The shaft 18, which, in this case, is in the form of a tubular shaft, can receive in its section adjacent to the free end a clip magazine 30, which holds a supply of the clips to be applied for these to be fed singly to the applying tool 20.

By actuating the handle 12, a clip 32 can be advanced from the clip magazine 30 to the applying tool where it is received in a guided manner in receiving areas 34, 35 of the tool jaws 24, 25 and held in readiness for application to a portion of tissue, for example, a blood vessel.

The receiving areas 34, 35 are in the form of grooves into which the clip 32 can be slidingly introduced with its legs 36, 37. For details of the exact functioning of the feeding mechanism, reference is again made in full to the description of FIGS. 1 to 3 of DE 19603889.

At its distal end, the receiving area 34, 35 of the tool jaws 24, 25 is closed by a wall 38, 39, while at its end remote therefrom it ends, in each case, with a recess 40, 41, which, when the tool jaws 24, 25 are closed, receive a connecting region 44 of the clip 32, which connects the two legs 36, 37 of the V-shaped clip 32 to each other.

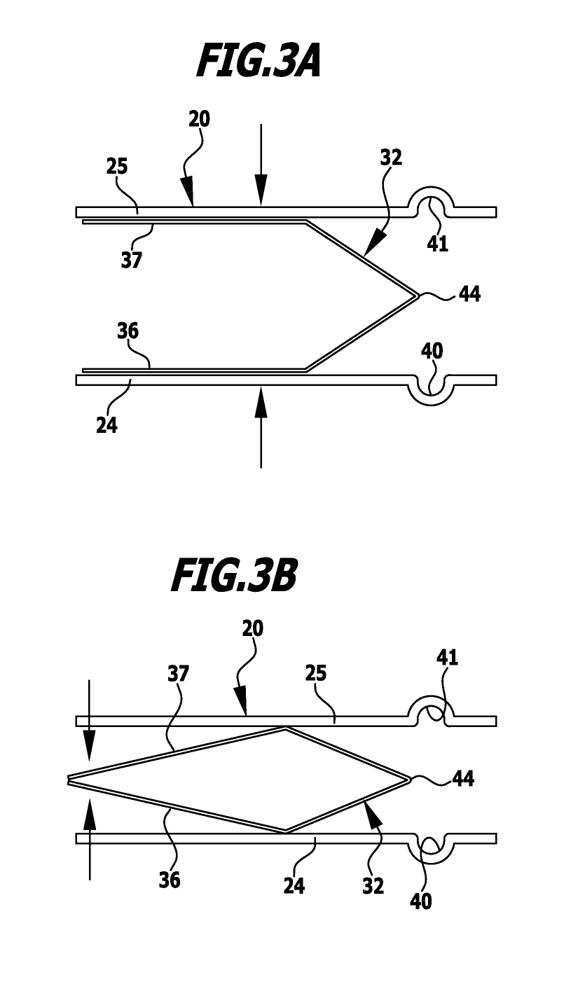

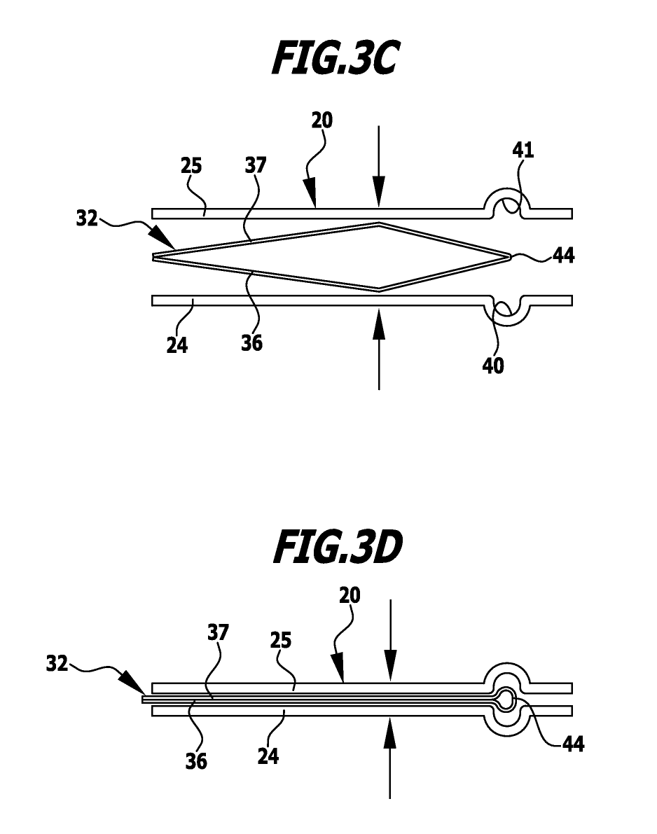

The operation of closing the clip with transfer of the tool jaws 24, 25 from the open, idle position to the closed position will be described with reference to the schematic FIGS. 3A-3D.

FIG. 3A again shows the clip 32 received by the tool jaws 24, 25 in the tool 20. The two tool jaws 24, 25 are subsequently transferred from the open, idle position in FIG. 3A to a closed position in FIG. 3D by a closing movement, indicated by the two arrows in FIG. 3A.

The two legs 36, 37 are first pivoted about the connecting region or apex 44 and are thereby brought closer together. This situation is shown in FIG. 3B, in which the pivotal movement of the legs 36, 37 has brought their free ends into abutment with each other.

When the closing operation is continued, as shown in FIGS. 3C and 3D, the two legs 36, 37 then undergo deformation, and a distinct bulge of material occurs at the connecting region 44, which requires an increased expenditure of force for further closure of the clip.

Owing to the inventive configuration of the receiving areas of the tool jaws 24, 25, the clip 32 can be closed completely without the sections of the legs 36, 37 immediately adjacent to the connecting region 44 having to be made to abut directly against each other. Rather, the recesses 40, 41 of the tool jaws 24, 25 following the receiving areas 34, 35 allow the clip legs adjacent to the connecting region to remain held at some distance from each other and to not be fully compressed. Therefore, the gap dimension obtained in the connecting region is larger than the gap dimension required between the legs.

As a result, a smaller bulge of material is created, and the legs 36, 37 of the clip can be brought more easily into their final closed position and to the gap dimension prescribed therefor of, for example, approximately 0.25 mm.

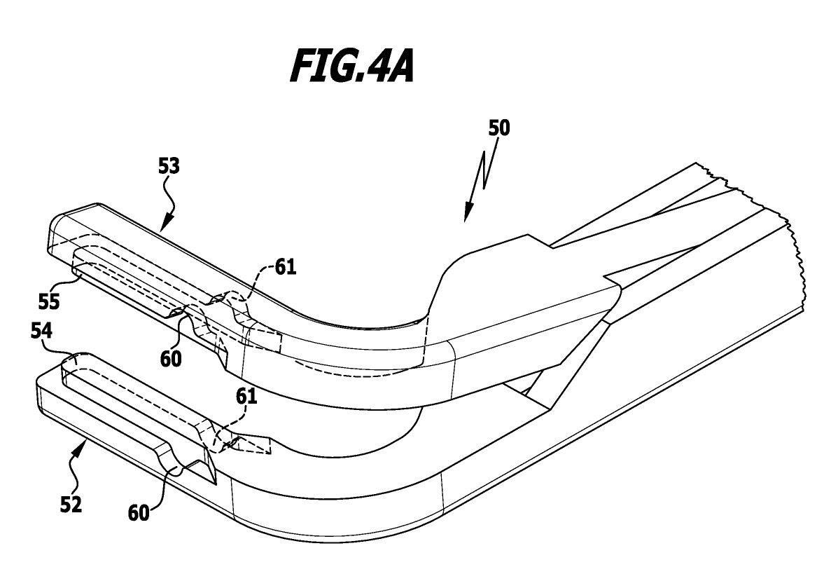

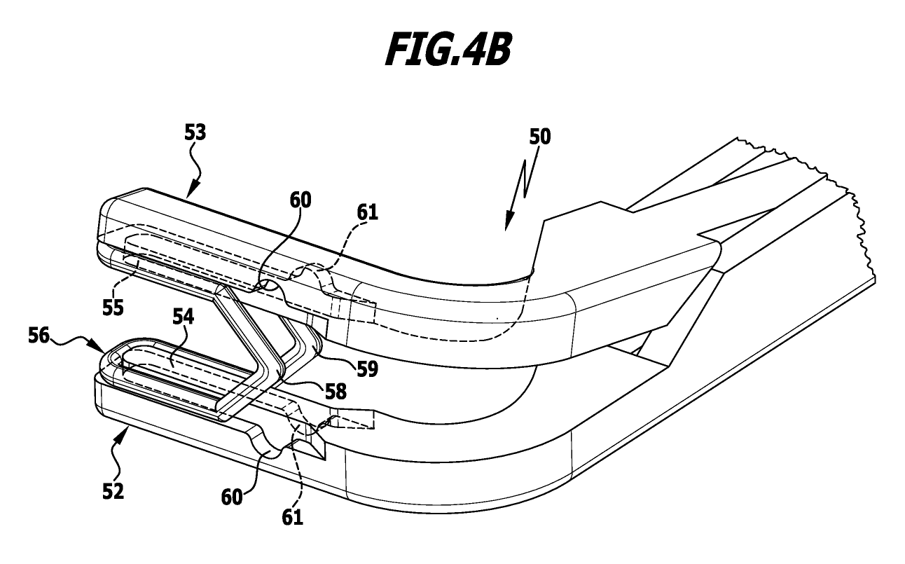

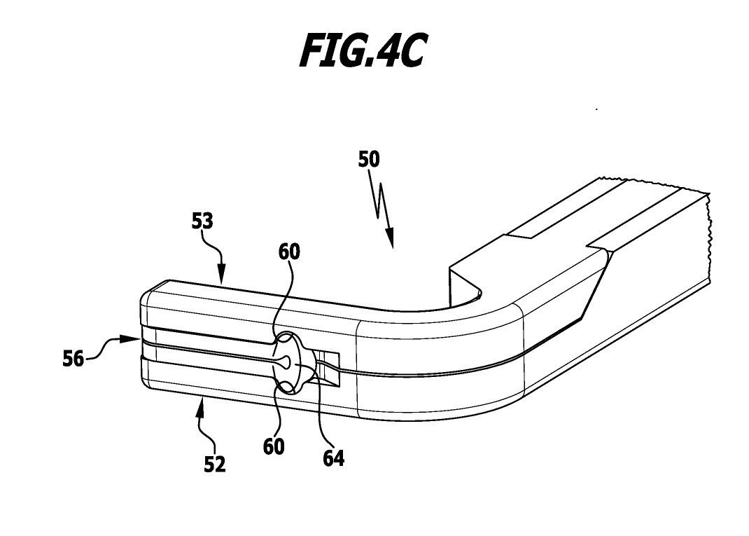

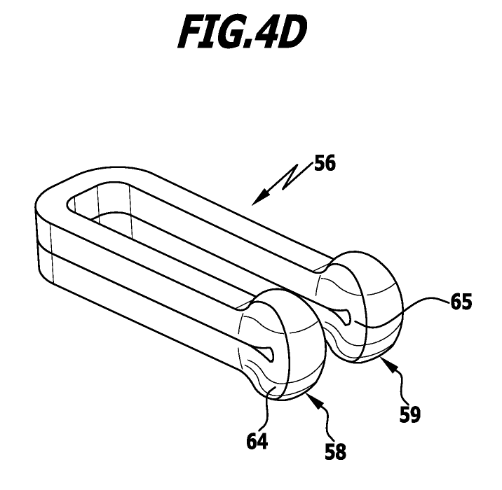

The operation shown schematically in FIGS. 3A to 3D is shown again in detail and in conjunction with applying a double-shank clip 56 with reference to an alternative embodiment of a surgical clip applicator and its applying tool 50 in FIGS. 4A to 4D.

The applying tool 50 of FIGS. 4A-4C differs from the applying tool 20 of FIGS. 1 and 2 in that the tool jaws are bent at an angle of approximately 90° in relation to the handle (not shown), and in that instead of a groove, the receiving areas of the tool jaws 52, 53 are provided with projections 54, 55 extending in the longitudinal direction of the tool jaws 52, 53 and serving to guide a so-called double-shank clip 56, the free clip ends of which are connected to each other.

Set back from the free end of the tool jaws 52, 53, recesses 60, 61 are provided in the sections in which a connecting region 58, 59 of the double-shank clip 56 comes to rest when the clip 56 is closed. The recesses 60, 61 prevent the connecting regions 58, 59 from having to be compressed to the same gap dimension as the clip legs when the double-shank clip 56 is closed.

The situation where the clip 56 is completely closed in the applying tool 50 is shown in FIG. 4C. In enlarged representation, FIG. 4D shows the closed double-shank clip 56 again where a bulge 64, 65 can be seen in the connecting region 58, 59.

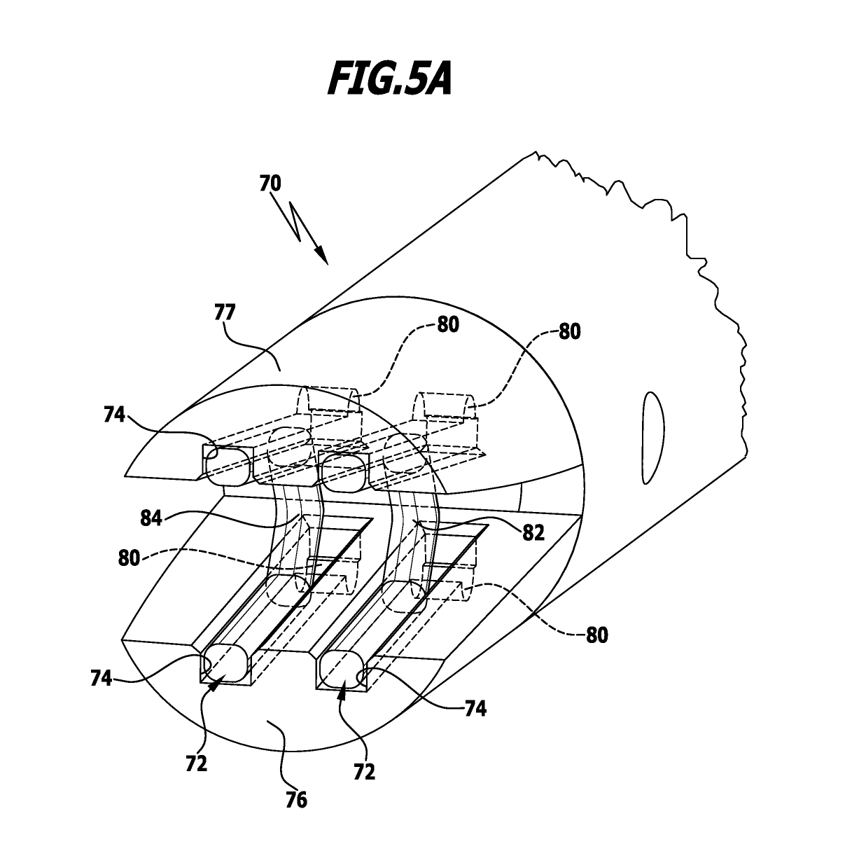

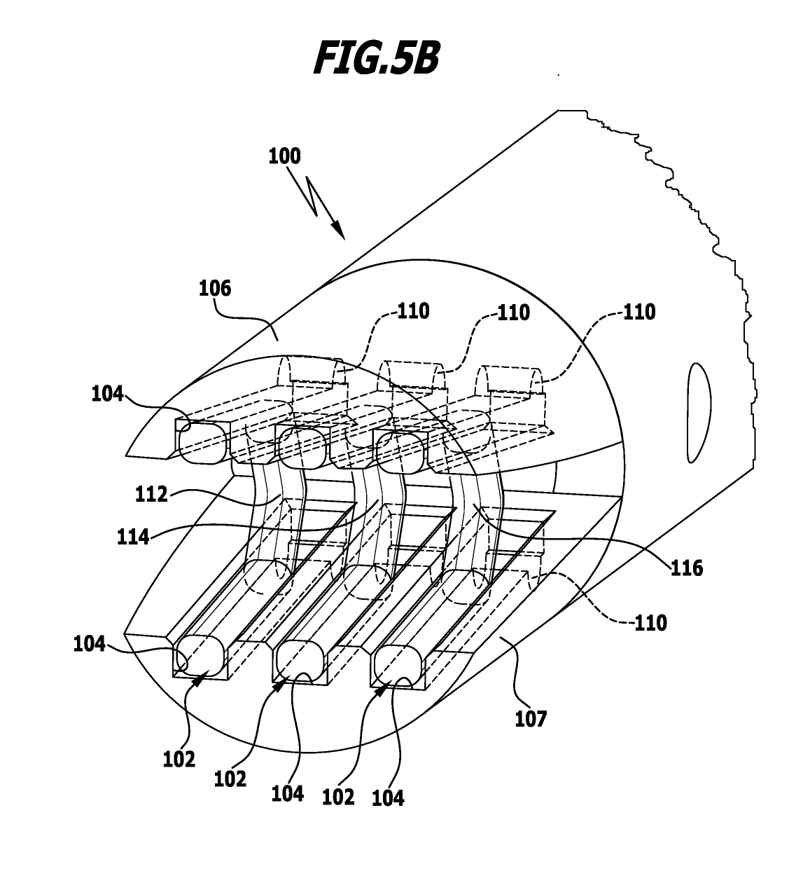

Two further alternative embodiments of the surgical clip applicator in accordance with the invention with applying tools 70 and 100, respectively, in which a double-shank clip 72 and a triple clip 102 are received in two grooves 74 and three grooves 104, respectively, are shown in FIGS. 5A and 5B.

The grooves 74; 104 of the tool jaws 76, 77; 106, 107 of the applying tools 70 and 100, respectively, function as receiving areas which widen into recesses 80; 110, so that the connecting regions 82, 84; 112, 114, 116 of the free legs of the double-shank clip and triple clip 72; 102 can escape and be received there during the closing operation, so that the connecting regions need not be compressed to a gap dimension prescribed for the clip legs.

In the case of guidance of the clips in grooves as receiving areas, it is, in general, advantageous if the recesses which receive the connecting regions of the free legs of the clips also widen in the transverse direction, so that the formation of the bulge at the sides of the individual clips (cf. FIG. 4D) can occur without restraint. This further facilitates the operation of closing the clip.

1. Surgical clip applicator for U-shaped or V-shaped clips having two legs connected to each other by a connecting region, said applicator comprising a handle, an adjoining shaft and at the free end of the shaft an applying tool, which is actuatable with the handle and has two tool jaws which are transferable from an open, idle position to a closed position when actuated and each define a receiving area for the clip to be applied, the receiving areas for the clip to be applied each comprising an abutment surface for one of the legs, which extends at least over part of the length of the legs, and a recess in which the connecting region of the legs of the clip is arranged when the tool jaws are in the closed position.

2. Clip applicator in accordance with claim 1, wherein the abutment surfaces have a setback in the area of the free ends of the legs.

3. Clip applicator in accordance with claim 2, wherein the abutment surfaces are of such dimensions that the legs abut only with a middle section against the tool jaws when the tool jaws are in the closed position.

4. Clip applicator in accordance with claim 3, wherein the abutment surfaces are of such dimensions that the legs abut over approximately a third of their length or more, in particular, half of their length or more, when the tool jaws are in the closed position.

5. Clip applicator in accordance with claim 4, wherein the abutment surfaces are of such dimensions that the legs abut over approximately two thirds of their length or more when the tool jaws are in the closed position.

6. Clip applicator in accordance with claim 1, wherein the recess has a depth perpendicular to the closing direction of the clip leg, which corresponds approximately to the diameter or thickness of a clip leg or more.

7. Clip applicator in accordance with claim 6, wherein the length of the recess parallel to the longitudinal direction of the clip leg corresponds approximately to twice the diameter or thickness of a clip leg or more.

8. Clip applicator in accordance with claim 7, wherein the length of the recess corresponds approximately to four times the diameter or thickness of a clip leg or less.

9. Clip applicator in accordance with claim 1, wherein the abutment surfaces comprise a guide element.

10. Clip applicator in accordance with claim 9, wherein the guide element comprises a groove.

11. Clip applicator in accordance with claim 9, wherein the guide element comprises a projection.
